(19) United States
(12) Patent Application Publication
(10) Pub. No.: US 2023/0186783 A1
(43) Pub. Date: Jun. 15, 2023

(54) A COMPUTER IMPLEMENTED METHOD FOR ESTIMATING A READING SPEED OF AN INDIVIDUAL

(71) Applicant: Novartis AG, Basel (CH)

(72) Inventors: Bjoern BORNKAMP, Weil am Rhein (DE); Daniel François Claude LORAND, Dietwiller (FR); Purvi Kishor PRAJAPATI, Greenwood, IN (US); John Weldon Seaman III, Mansfield, TX (US)

(21) Appl. No.: 17/995,753

(22) PCT Filed: Apr. 26, 2021
(86) PCT No.: PCT/IB2021/053428

§ 371 Date: Oct. 7, 2022

Related U.S. Application Data
(60) Provisional application No. 63/018,620, filed on May 1, 2020.

(51) Int. Cl. G09B 17/04; G09B 5/02

(52) U.S. Cl. CPC G09B 17/04; G09B 5/02

(57) ABSTRACT

A computer-implemented method for estimating a reading speed of an individual. The method comprises the steps of: providing a reading speed model, based on a reading speed function comprising a set of parameters and a set of metrics; determining a printed stimulus to be presented to the individual by way of a reading run of a reading test and determining one or more stimulus features associated therewith; controlling an administration of the reading test to the individual based on the one or more determined stimulus features; receiving reading speed observation data based on the presented printed stimulus and corresponding to one or more responses made by the individual in the reading test.

### TECHNICAL FIELD

The present invention relates to a computer-implemented method for estimating a reading speed curve of an individual. In particular, the computer-implemented method according to the present invention outputs an estimate of a reading speed function for an individual obtained by administering a computer-based reading test to the individual, in the form of determined printed stimuli presented to the individual, thus ultimately characterizing the reading speed of the individual over a range of one or more features of the presented printed stimuli.

The present invention also relates to a computing system, designed to carry out the method, wherein the computing system comprises a computing device including one or more processors; one or more input and/or output elements; memory; and one or more programs stored in the memory including instructions for implementing the method.

The present invention further relates to a non-transitory computer-readable storage medium storing one or more programs configured to be executed by one or more processors of an electronic device with one or more input and/or output elements, the one or more programs including instructions for carrying out the above mentioned computer-implemented method.

The computer-implemented method according to the present invention can be typically run on mobile computing platforms, for instance by being incorporated in digital mobile application modules or App modules. Such mobile application modules can be useful for functionally testing the visual ability of individuals having some ocular disease, for instance geographic atrophy, without the need for the individuals to attend clinical visits. The computer-implemented method according to the present invention can therefore also be advantageously employed in the context of decentralized clinical trials or for disease monitoring from remote.

### BACKGROUND ART

Reading speed is a functional measure of visual ability assessed over a range of print sizes. A common reading test is the MNREAD test developed in the late 1980s. It is a continuous text reading test presenting sentences to a tested individual, with specific rules for the presented sentences. Namely, these sentences must be of a certain length across three lines. The text size, even the size of the white space, is specified. The difficulty of the words in the sentences is around a third grade level, providing a test that can be used by children as well as by adults. It was originally developed as a paper test. Reading speed for this test is a function of how fast a patient can read a presented sentence of a specified size and how many errors are made during the reading. The test proceeds from the largest print size to the smallest print size a patient can read. There are different stopping rules, but one such rule would be to stop on the sentence in which no words are read correctly. The paper test requires an observer using a stopwatch to capture the length of time it takes to read a sentence; and to count the number of errors a patient makes.

Current paper-based reading performance charts are often cumbersome to use, owing to the fact that manual time measurement, sentence presentation and error recording have to be undertaken simultaneously by a supervising examiner. Additionally, reading performance metrics are determined by the examiner by plotting reading performance data graphically. This is a time-consuming process, prone to mistakes, which is better suited to automation. Any metrics determined subjectively by the examiner are liable to a high degree of variability; for instance, data around a critical print size, alternatively designatable as CPS, corresponding to the print size after which reading speed declines rapidly, are subject to relevant noise and are likely to be misjudged.

As an alternative to paper charts, computerised reading systems have been used for reading speed assessment, but display technology on personal computers was in the past not sufficiently advanced to be able to test a wide range of print sizes at typical reading distances. Also, personal computers are not as portable as paper charts.

Lately, the technical performance of displays of mobile computing platforms, such as mobile phones and tablets e.g. the iPad, has proved suitable for the testing of visual functions. High-resolution displays in mobile phones and tablets can nowadays render small text sizes perfectly, or at least adequately well for the intended purpose, at standard reading distances, such as 40 cm.

By way of example, an iPad version of the MNREAD test has been developed and validated. A timing clock starts and stops with taps on the tablet screen.

However, current reading speed tests do not achieve to fully exploit the potential for automation of such mobile computing platforms. Particularly, known reading speed methods implemented on mobile computing platforms do not allow the tested individual, or patient, to take the test fully autonomously at home, without the assistance of an observer. Moreover, available solutions do not optimally remove human reaction time from the recording of reading time and are thus disadvantageously affected by a relatively high test-retest variability.

In addition to that, currently available digital reading speed tests fail to exploit efficiently the processing power of mobile computing devices on which they are carried out, resulting in relatively long waiting times for calculations to be implemented and in yet scarce recording precision. The duration of conventional reading tests is nowadays rather inconveniently long, as these tests substantially go through all print sizes and are unable to carry out an intelligent selection thereof.

Ultimately, currently available methods for estimating the reading speed of an individual do not lend themselves to decentralized clinical trials wherein the tested individuals can autonomously record their own reading performance and/or to remote disease monitoring.

Therefore, there is a need for a computer-implemented method to provide a consistently repeatable, reliable, precise and fast estimation of a reading speed of an individual, which can be executed on a computing device, preferably a mobile computing device, when the individual self-administers a reading test, in full or in partial autonomy, making the presence of a health-care provider such as a doctor unnecessary or optional, and which enables a substantially automatic determination of reading metrics of a reading speed function characterizing the reading speed of the individual.

### DISCLOSURE OF THE INVENTION

According to the invention, this need is settled by a computer-implemented method for estimating a reading speed of an individual as it is defined by the features of independent claim 1; by a system as it is defined by the features of independent claim 27; and by a non-transitory computer-readable storage medium as it is defined by the features of independent claim 28. Further embodiments are herein described, for example, in the dependent claims.

In particular, the invention deals with a computer-implemented method for estimating a reading speed of an individual, or a corresponding reading speed curve. The method can be implemented on a computing device including one or more processors and one or more input and/or output elements.

The method comprises a step of providing a reading speed model, based on a reading speed function comprising a set of parameters and a set of metrics.

Specific metrics that can be used in the present reading speed model can be, for instance, the Maximum Reading Speed, also designatable with the acronym MRS, which can be defined as an individual's reading speed, when reading is not limited by print size; and the Critical Print Size, also designatable as CPS, which can be defined as the print size below which reading speed declines rapidly.

Further to the reading speed model provision, a printed stimulus is determined, to be presented to the individual by way of a reading test. In the context of the present invention, the term “printed” can broadly indicate a graphical rendition of a stimulus which is not limited to a transfer thereof to a paper substrate, but preferably encompasses a graphical display on a surface for viewing, such as on a screen of a mobile computing device. Accordingly, even (hand-)written messages or notes which are subsequently rendered on a display of a computing device can be encompassed.

In various embodiments of the method according to the present invention, the printed stimulus can take the form of a sentence. A whole sentence can be advantageously designed to contain more information, and be thus more meaningful to the implementation of the method, than a graphical rendition limited to a comparable number of disconnected single words. The presentation of whole sentences can therefore enhance the efficiency of carrying out the present method, resulting in quicker execution times thereof.

Alternatively, the printed stimulus can take the form of one or more graphical elements which are representative of generic text, including a word or a sequence of single, uncorrelated words; and/or illustrating an object, such as images or designs; and/or a combination thereof.

The method according to the present invention further comprises a step of determining one or more stimulus features associated with the printed stimulus. In various embodiments, the most representative stimulus feature can be selected as the size of the printed stimulus. Alternatively, or in addition thereto, stimulus features can comprise one or more of font style; font style; display background light and/or colour; contrast; general formatting of text and/or of images, including spacing etc.

Moreover, the method according to the present invention further comprises a step of controlling, for instance by way of processor functions of the computing device it is implemented on, an administration of the reading test to the individual based on the one or more determined stimulus features. By way of example, the determined printed stimulus to be presented to the tested individual can be selected according to optimization criteria controlling the print size thereof.

The reading speed function is configured to characterize the reading speed of the tested individual over a range of the one or more stimulus features of the presented printed stimulus. The reading speed function thus can represent a reading curve for the tested individual, and it can mimic, as a result, at least part of a run of the administered reading speed test.

The estimate of a reading speed of an individual can be output by embodiments of the present invention ultimately as a reading curve for the tested individual, spanning over the tested range of print sizes of the presented printed stimuli.

In various embodiments, the printed stimulus to be presented can be selected according to optimization criteria, preferably in order to maximize information on the reading curve to be estimated, as it will be more in detail described in the body of the specification.

The set of parameters of the reading speed model and the set of metrics incorporated in the reading speed function are preferably estimated by subsequently administering the test to the individual.

An exemplary method according to the present invention further comprises a step of receiving reading speed observation data based on the presented printed stimulus and corresponding to one or more responses made by the individual in the reading test.

Reading speed observation data can comprise reading times reported and/or reading errors counted for each run of the test, as it will be more in detail described in the following.

In various embodiments, the one or more responses made by the individual in the reading test are assessed by voice recognition software, e.g. both for reporting reading time and for counting errors. Voice recognition thus employed would allow the patient to take the test at home, without need for an observer. Removing human reaction time from the recording of reading time also advantageously improves test-retest variability. Alternatives to voice recognition are also envisaged. Voice recognition can also be substituted, in part or in total, with manual entry of registered data, for instance recorded by a health-care professional.

An exemplary method according to the present invention comprises an adaptive phase of fitting the reading speed model to the received observation data. The set of parameters and/or of metrics of the reading speed model and/or the set of stimulus features can be updated during the adaptive fitting process, based on current reading speed observation data. Thus, the estimated reading speed of the tested individual is also consequently updated.

Within the adaptive phase, the steps of providing a reading speed model; of adaptively determining a printed stimulus to be presented to the tested individual; of controlling the administration of the reading test to the individual based on one or more features of the stimulus as determined; of receiving observation data and of fitting the reading speed model to the received observation data are iterated, according to stopping criteria which dictate the conditions under which the iteration can be halted. As a result of this iteration, the estimated reading speed for the individual is refined through a plurality of subsequent administrations of reading runs of the reading test.

The method according to the present invention is particularly designed to boost the adaptive phase, in a way that the duration of the tests successively submitted during such phase is advantageously shortened and a higher precision is ultimately achieved in estimating the reading speed of the tested individual.

To this purpose, the method according to the present invention is conceived in a way that, before the adaptive phase, providing the reading speed model comprises a step of mapping out the reading speed function for the currently tested individual. More precisely, this operation of mapping out the reading speed function best characterizing the tested individual is executed by a preliminary administration of reading runs of the reading test to the individual, based on presentation of printed stimuli having predetermined values of the one or more features; and by consequently receiving reading speed data to obtain a corresponding number of points on an individual-specific reading curve. The ultimate goal of this step is to provide initial values along the reading curve for the tested individual.

By doing so, the starting point for the successive adaptive phase does not rely on baseline data extracted from a generic set of individuals, for instance individuals with a healthy reading performance. Rather, the present method is from the initial phase customized to the specific individual being tested, as it relies on individual-specific observed data, used to preliminarily map out the reading curve for the currently tested individual. In particular, the choice of points obtained, which are representative of points on an individual-specific reading curve, can be purposely steered or targeted in a way that the actual shape and the position of the reading curve is best approximated.

In embodiments of the invention, providing the reading speed model, and more specifically providing initial values along the reading curve, comprises a step of defining the range of the one or more stimulus features and identifying a pre-defined number of predetermined values within the range of the one or more stimulus features to divide the range into corresponding portions. Thus, the range comes to have a minimum predetermined value of each stimulus feature and a maximum predetermined value of each stimulus feature.

In various embodiments, the portions of predetermined values are substantially equal.

In various embodiments wherein one of the features of the presented printed stimulus is print size, providing the reading speed model comprises a further step of calculating, by the reading speed function, adjusted reading speeds for each of the identified predetermined values of print size.

Moreover, for such embodiments, the method can comprise a step of counting the number of errors which the individual makes when reading at each of the identified predetermined values of print size. Incidentally, the adjusted reading speed can be defined as the reading speed after taking into consideration the number of errors the individual made while reading at a given print size.

In the context of the present invention, by the term “adjusted reading speed” it will be meant a reading speed calculated according to the following formula:

$$Adj . Reading Speed = 60 ( \frac{10 - \# errors}{Reading Time} )$$

wherein #errors stands for number of errors.

In embodiments of the invention, the reading speed function corresponds to a logistic model, according to the following function:

$$f ( {x}_{j} ) = \frac{ϕ}{1 + exp [ - a ( {x}_{j} - b ) ]}$$

wherein:
ϕ is a metric corresponding to the maximum reading speed on the reading curve for the individual, that is, the individual's reading speed when reading is not limited by print size;
a is the rate of change from 0 to ϕ on the reading curve; and
b is an inflection point of the reading curve.

By way of example, in a range wherein the minimum print size is set to −0.10 log MAR and the maximum print size is set to 1.3 log MAR, five initial print sizes can be selected and for each one of them observation data can be obtained, which correspond to respective adjusted reading speeds for the tested individual and are representative of respective points on a reading curve specific to the tested individual.

With reference to the steps of calculating, by the reading speed function, adjusted reading speeds for each of the identified predetermined values of print size and of counting the number of errors, the process can start by executing a step of testing the individual by presenting printed stimuli starting at the largest predetermined value of print size of the range, wherein the initial print size value is the maximum i.e. largest print size value of the range. Referring to the above example, the tested individual can start the reading test at the largest print size, 1.30 log MAR.

As a result of the above step, reading speed observation data can be received to obtain a first observed value of the initial reading speed. Accordingly, a point on the reading curve can be obtained corresponding to such largest predetermined value print size of the range.

Providing the reading speed model can further comprise the step of providing check criteria based respectively on a cutoff error value for the number of errors which the individual makes at a given print size value; and on a cutoff speed value for the adjusted reading speed at a given print size value.

In some embodiments, the cutoff speed value for the adjusted reading speed at a given print size value can be set to a percentage of the first observed value of the initial reading speed.

The check criteria can be used to assess whether a preliminary estimate of the reading curve for the tested individual can serve as a base to pass to the successive adaptive phase abovementioned. That is, the check criteria can be used to assess whether data points obtained from observation in the preliminary phase can be used for a successive fitting of the reading curve.

To this purpose, in some embodiments the method comprises the step of comparing respectively the number of errors which the individual makes at a given print size value to the cutoff error value. A first check criterion is met if the number of errors is less than, or equal to, the cutoff error value. The cutoff error value can be considered a maximum error threshold. Moreover, the check function can be complemented with a step of comparing the adjusted reading speed at a given print size value to the cutoff speed value, establishing that a second check criterion is met if the adjusted reading speed is equal or above the cutoff speed value. The cutoff speed value can be considered a minimum speed threshold.

In some embodiments, if both check criteria are met, a further step follows of reducing the print size value to the next smaller predetermined value. Under these conditions, the individual is newly tested by presenting to him printed stimuli at the next smaller predetermined value of print size. Consequently, a step is instructed of comparing the counted number of errors which the individual makes at the next smaller predetermined value of print size to the cutoff error value. Also, the method comprises a step of comparing the calculated adjusted reading speed at the next smaller predetermined value of print size to the cutoff speed value.

If it is then verified that no further smaller predetermined value of print size exists, the reading speed observation data and the errors made by the individual when reading at each of the identified predetermined values of print size are stored for completing the provision of the reading speed model, in the above described preliminary phase preparatory to the successive adaptive phase.

Otherwise, if either of the check criteria is not met, some embodiments of the method according to the present invention comprise the step of increasing the print size value by a predefined amount with respect to the current print size value, if this is given or possible within the range of predetermined values of print sizes. There follows a step of testing the individual by presenting printed stimuli at the corresponding increased print size value. Based on the observed data, then the method instructs a step of comparing respectively the number of errors which the individual makes at the corresponding increased print size value to the cutoff error value, and a step of comparing the adjusted reading speed at corresponding increased print size value to the cutoff speed value. By way of example, the print size value can be increased stepwise by predefined amounts such as +0.05 log MAR or +0.1 log MAR.

If, after any step increase of the print size value as above described, both check criteria come to be newly met, the method according to the present invention preferably stores the reading speed observation data and the errors made by the individual when reading at each of the values of print size, for completing the provision of the reading speed model. Otherwise, if either of the check criteria is not met, the print size value is further increased by a predefined amount, such as +0.05 log MAR or +0.1 log MAR, with respect to the current print size value, up to the last print size value at which both check criteria were previously met.

In various embodiments, if either of the check criteria is not met already when testing the individual by presenting printed stimuli starting at the largest predetermined value of print size of the range, the testing of the individual at the largest predetermined value of print size of the range is repeated. If either of the check criteria is again not met, the adaptive phase is not initiated.

In some embodiments, irrespective of observation scenario/outcome whether the check criteria were met or whether the check criteria were not met, the method according to the present invention comprises a step of storing the reading speed observation data and the errors made by the individual when reading at each of the values of tested print sizes in a final data set, in order to complete the provision of the reading speed model.

After the completion of the preliminary phase, preparatory to the successive adaptive phase, in some embodiments the first iteration step of fitting the provided reading speed model to received observation data is based on fitting the provided reading speed model to the observation data obtained as above explained and stored in a final data set. Thus, the set of parameters and/or of metrics of the reading speed model is updated based on the stored reading speed observation data.

Moreover, in some embodiments, fitting the provided reading speed model to received observation data corresponding to subsequent administrations of reading runs of the reading test to the individual comprises the step of defining a fitting function, comprising the reading speed function. The log posterior for the defined fitting function is then optimized, to adaptively estimate the set of parameters and the set of metrics of the reading speed model. In general, the log posterior density function for the parameters is the sum of data log-likelihood function multiplied by the prior log-density function. Ultimately, the set of parameters and the set of metrics of the reading speed model thus estimated are used to calculate fitted adjusted reading speeds for each available print size.

In some embodiments, at the beginning of the adaptive phase, and after each successive presentation of stimuli, the parameters of the reading speed model, for instance a logistic model, can be estimated as follows.

The prior distribution π(ϕ, a, b) for the model parameters ϕ, a and b, representing any prior knowledge or expectations about the model parameters, can be assumed to be a multivariate normal distribution, with mean vector μ and covariance matrix Σ, according to the following expression:

log(ϕ),log(a),b˜N(μ,Σ)

The prior distribution π(σ) for the standard deviation of the data model can be assumed to be a log-normal distribution with mean μ_{σ} and variance τ² according to the following expression:

log(σ)˜log normal(μ_{σ},τ²)

This represents any prior knowledge or expectations about the model parameters, before evidence of yet newly calculated observed data is taken into account. We choose what is referred to as a relatively non-informative prior distribution for the parameters, but the model is flexible to allow for more historical data/expert opinion to be incorporated into the prior.

By adopting a Bayesian approach, the data is modeled using a Normal distribution and the log-likelihood function

l(ϕ, a, b, σ)=Σⱼ₌₁ⁿ log (ƒ_{y}(yⱼ)) can be calculated. In the above equation, yⱼ can be set as the adjusted reading speed at the corresponding print size, xⱼ, wherein yⱼ can be expressed in word per minute, or WPM.

yⱼ can be assumed to have a normal distribution according to the following exemplary expression:

$${y}_{j} ∼ N ( \frac{ϕ}{1 + exp [ - a ( {x}_{j} - b ) ]} , σ )$$

wherein the mean is defined by the logistic model equation already introduced above, in connection with the reading speed function ƒ(xⱼ), formulated as

$$\frac{ϕ}{1 + exp [ - a ( {x}_{j} - b ) ]}$$

and σ is the variability of the reading speed measurement, taking into account that at any print size, the same tested patient reading the same sentences over and over would have somewhat varying reading speeds.

yⱼ substantially characterizes the reading curve which the reading speed model according to the present invention intends to produce.

The log-posterior is a sum of the log likelihood and the log priors. A negative log posterior distribution, proportional to

−l(ϕ,a,b,σ)−log(π(ϕ,a,b))−log(π(σ))

can be calculated, in order to subsequently optimize the log posterior distribution by identifying the parameter estimate combination that maximizes the log posterior distribution or, conversely, minimizes the negative log posterior distribution. Such optimization routine will optimize multiple-argument reading speed function ƒ(xⱼ).

Parameter values of ϕ, a and b that maximize the log posterior distribution are then used as updated estimates for ϕ, a, and b.

Metric estimates such as Maximum Reading Speed, or MRS, and Critical Print Size, or CPS, are then functions of these parameter estimates and can be calculated and updated accordingly.

In some embodiments, the stopping criteria for the iterations of fitting the provided reading speed model to received observation data are based on cutoff values for the estimated metrics, preferably on the posterior standard deviation, or standard errors, for the respective estimates of the metrics employed, or on a combination of the standard errors for such metrics.

Thus, the stopping criteria can be based on cutoff values related to the maximum reading speed, or MRS, and the critical print size, or CPS.

In some embodiments, fitting the provided reading speed model to received observation data corresponding to subsequent administrations of reading runs of the reading test to the individual comprises the step of controlling the print size of the next/subsequently administered printed stimulus, so that a corresponding point is obtained on the reading curve in a targeted position. This adaptive step can be reiterated, up to when the stopping criteria are met and/or the maximum number of sentences to be presented has been reached.

In some embodiments, choosing the corresponding point on the reading curve lies within a subset of a parameter or metric space for one of the parameters or metrics of the reading speed model, such as for the maximum reading speed, or MRS.

In some embodiments, the print size of the next/subsequently administered printed stimulus can be selected in blocks of e.g. three or five distinct print size values, so that corresponding, distinct points are obtained in respective targeted positions of the reading curve. For instance, the targeted positions of the reading curve can be on an elbow, on a slope, and/or on a plateau of the reading curve. These targeted positions can correspond to respective percentages of the maximum reading speed, or MRS. Specific tests carried out in the context of the present invention have proven that blocks of three distinct print size values can be sufficient.

Preferably, the range of print sizes is chosen in compliance with the resolution capability of the device on which the reading assessment is being used.

Ultimately, fitted adjusted reading speeds can be calculated for each of the print sizes comprised in the data set; accordingly, fitted reading parameters and metrics can be calculated based on the reading speed function which has been fit to the received observation data.

The present invention also refers to a system comprising a computing device including one or more processors; one or more input and/or output elements; memory; and one or more programs stored in the memory including instructions to execute the method above described. Preferably, in order to allow more flexibility from remote and enhance the feasibility of decentralized clinical trials, a mobile, hand-held computing device can be used, for instance a tablet or a smart-phone.

The present invention also refers to a non-transitory computer-readable storage medium storing one or more programs configured to be executed by one or more processors of an electronic device with one or more input and/or output elements, wherein the one or more programs are designed to include instructions to execute the method above described.

### BRIEF DESCRIPTION OF THE DRAWINGS

The method and the system and according to the invention are described in more detail herein below by way of exemplary embodiments and with reference to the attached drawings, in which:

FIG. 1 illustrates an exemplary known reading curve, wherein reading speed is mapped out as a function of stimuli print size for a tested individual, highlighting some of the metrics which are referenced and used in some embodiments of the method for estimating a reading speed of an individual according to the present invention;

FIG. 2 illustrates an exemplary sequence of phases in an implementation of a method for estimating a reading speed of an individual according to the present invention;

FIG. 3 illustrates an exemplary work-flow of a preliminary phase of an embodiment of the method for estimating a reading speed of an individual according to the present invention; and

FIG. 4 illustrates an exemplary work-flow of an adaptive phase of the embodiment of FIG. 3, wherein the adaptive phase is subsequent to the preliminary phase.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

The following description sets forth exemplary systems, devices, methods, parameters, and the like. It should be recognized, however, that such description is not intended as a limitation on the scope of the present disclosure but is instead provided as a description of exemplary embodiments. For example, reference is made to the accompanying drawings in which it is shown, by way of illustration, specific example embodiments. It is to be understood that changes can be made to such example embodiments without departing from the scope of the present disclosure.

As used herein, the terms “subject” or “individual” are equivalent to the term “patient” and refer to a mammalian organism, preferably a human being, who may be diseased with the condition (e.g., disease or disorder) of interest and who may benefit biologically, medically, or in quality of life from treatment for the condition.

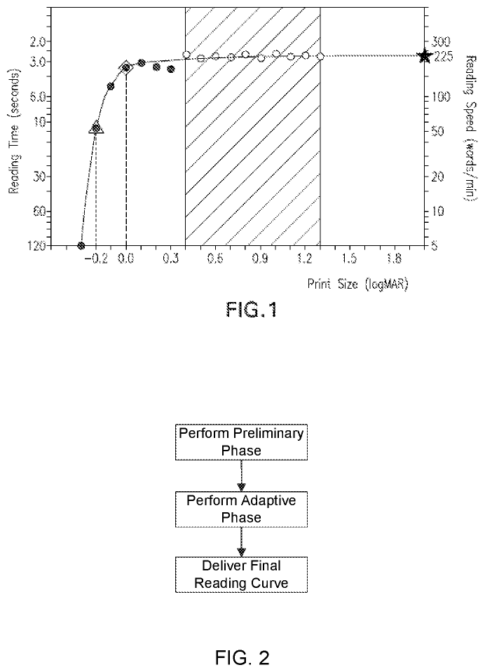

With initial reference to FIGS. 1 and 2, the computer implemented method for estimating a reading speed of an individual according to the present invention substantially aims at characterizing the reading speed of the individual over a range of one or more stimulus features, such as print sizes, of printed stimuli which are subsequently presented to an individual during respective runs of a reading test.

FIG. 1 is an example of a prexisting reading curve, drawn from “Baseline MNREAD Measures for Normally Sighted Subjects From Childhood to Old Age” by Calabrese et al., published in “Investigative Ophthalmology & Visual Science”, 2016. In the Figure, the x-axis is print size as measured by log MAR, and the Y axes show reading time in seconds and reading speed in words per minute. A minimum reading acuity is shown to be −0.2 log MAR whilst a critical print size (below which reading speed is impaired) is 0.0 log MAR. The hatched band, between 0.4 and 1.3 log MAR represents a range of the ten largest print sizes on the standard MNREAD chart.

The ultimate goal of the method according to the present invention is estimating a reading curve representative of the reading performance of the tested individual, mimicking at least part of a run of the administered reading speed test, such as the reading curve shown in FIG. 1, wherein reading speed is mapped out as a function of stimuli print size for the tested individual.

As shown in FIG. 2, the method for estimating a reading speed of a tested individual according to the present invention overall comprises a sequence of steps performing a preliminary phase; a successive sequence of steps configured to perform an adaptive phase; and a final sequence of steps aimed at delivering a reading curve specifically obtained for the tested individual, in a form similar to the one shown in FIG. 1.

The gist of an administration of the reading test to the individual in the preliminary phase, based on presentation of printed stimuli having predetermined and targeted values of print sizes, is to preliminarily approximate the actual shape and position of the reading curve for the currently tested individual, in a way that the successive adaptive phase quickly converges to yield a reading curve best characterizing the reading performance of the tested individual.

In some embodiments a selection of print sizes are chosen in a preliminary phase of the method for estimating a reading speed of a tested individual according to the present invention, wherein for each of the print sizes observation data is obtained which correspond to respective adjusted reading speeds for the tested individual and are representative of respective points on a reading curve specific to the tested individual. In some embodiments, the five print sizes are selected, at equal points between −0.10 log MAR to 1.3 log MAR.

In connection with each of such predetermined values of print size, corresponding points representative of adjusted reading speeds of the tested individual have been obtained. The adjusted reading speeds can be calculated by a reading speed function. Also, the number of errors made by the tested individual when reading at each of the predetermined print sizes is counted and recorded.

For example, if the minimum print size is set to −0.10 log MAR and the maximum print size is 1.3 log MAR, with five initial points, the print sizes at which data are collected, both in terms of reading speed and errors made, are 1.3 log MAR, 0.95 log MAR, 0.60 log MAR, 0.25 log MAR, and −0.1 log MAR.

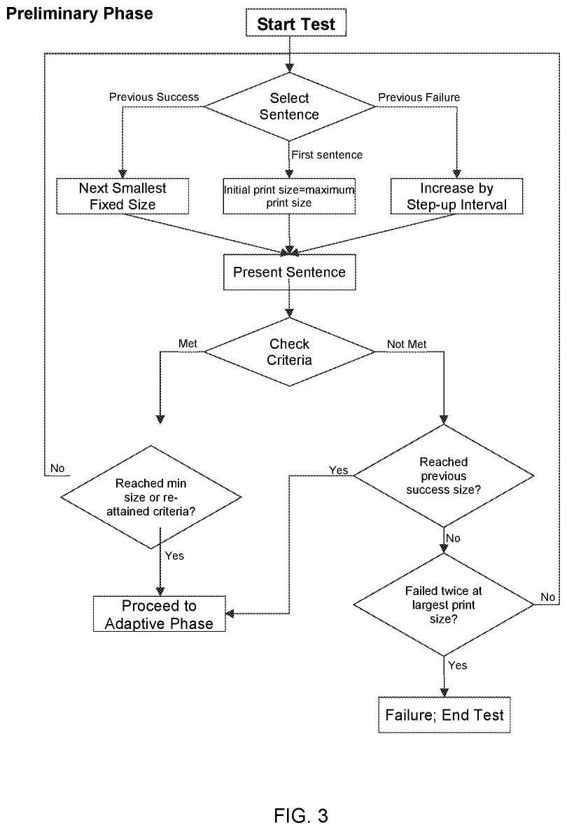

Reference will be made to the work-flow capturing the sequence of steps of the preliminary phase of FIG. 3, wherein the succession of steps has been previously explained in the disclosure of the invention. The reading speed at the maximum print size is referred to as the individual's initial reading speed. This is obtained by presenting printed stimuli to the tested individual starting at the largest predetermined value of print size of the range, namely 1.3 log MAR for the specific case exemplified. Corresponding reading speed observation data are received, to obtain a first observed value of the initial reading speed. A first point on the reading speed curve is obtained, resulting in an adjusted reading speed corresponding, for example, to a value of print size equal to 1.3 log MAR.

After obtaining the data at the initial, e.g. largest, print size, test criteria are checked. These test check criteria are based respectively on a cutoff error value for the number of errors which the individual makes at a given print size value; and on a cutoff speed value for the adjusted reading speed at a given print size value. For instance, the cutoff speed value for the adjusted reading speed at a given print size value can be set to a percentage of the first observed value of the initial reading speed at 1.3 log MAR.

The number of errors which the individual makes at 1.3 log MAR is compared to the cutoff error value. A first check criterion is met if the number of errors is less than or equal to the cutoff error value. If the tested individual makes more errors than the cutoff error value, then the tested individual has to read another sentence at the largest print size, e.g. at 1.3 log MAR. If the number of errors newly exceeds the cutoff error value, then the reading test ends for this tested individual and does not proceed to the subsequent adaptive phase. Both observations at the maximum print size are saved in a final data set.

If, instead, in this new observation the number of errors is less than the cutoff error value, then the reading speed of this observation becomes the initial reading speed and the test proceeds to the next print size, which is 0.95 log MAR in the given example.

If the tested individual reads the presented sentence at 0.95 log MAR and meets both check criteria, observations are added to the final data set and the test proceeds to the next smaller predetermined value of print size, namely 0.60 log MAR.

If at 0.60 log MAR the tested individual makes, for instance, more errors than the cutoff or reads slower than the cutoff speed value, this leads to the individual having to take the test with printed stimuli having a print size value increased by a predefined amount. Such predefined amount, or step-up interval, can be of +0.05 log MAR or of +0.1 log MAR, with respect to the current print size value. Thus, 0.95 log MAR becomes the new maximum print size, at which check criteria were met.

Therefore, the tested individual will have to take the test a print size of, e.g., 0.70 log MAR. If the observation data at 0.70 log MAR meets the check criteria, then the preliminary phase is deemed complete for the tested individual. If, however, the check criteria are not met at 0.70 log MAR, then the current print size value is further incremented by a predefined amount. Observation data are, for instance, obtained for a print size of the presented stimuli of 0.80 log MAR. If, once again, the observation does not meet the criteria, then a final observation is taken at 0.90 log MAR, which is below the new maximum print size at which the test was last successful, that is 0.95 log MAR.

After reading the sentence at 0.90 log MAR, and registering the relative observation data, the initial phase for the tested individual would end, since a newly incremented print size would equal or exceed the new maximum print size.

Irrespective of observation scenario/outcome, whether the check criteria were met or whether the check criteria were not met, the method according to the present invention can comprise the step of storing the reading speed observation data and the errors made by the individual when reading at each of the values of tested print sizes in a final data set, in order to complete the provision of the reading speed model achieved during the preliminary phase.

In the embodiment hereby exemplified, the minimum number of observations an individual can have is two, when the individual fails twice at the maximum print size, and the remainder of the test is not completed (i.e. the adaptive phase is not entered). Conversely, the minimum number of observations an individual can have in a successful preliminary phase is three.

As shown in FIG. 2, the preliminary phase is generally followed by the adaptive phase, then by acquisition of the final reading curve. The first iteration step of fitting the provided reading speed model to received observation data is based on fitting the provided reading speed model to the observation data obtained by implementing the preliminary phase and stored in the abovementioned final data set.

The first iteration step is then followed by further steps of fitting the provided reading speed model to received observation data corresponding to subsequent administrations of reading runs of the reading test to the individual. To this purpose, a fitting function is defined, comprising the reading speed function, and the log posterior for the defined fitting function is optimized, to estimate the set of parameters and the set of metrics of the reading speed model. The estimates for the set of parameters and/or of metrics of the reading speed model are adaptively updated, based on the reading speed observation data progressively received. The set of parameters and the set of metrics of the reading speed model thus estimated are used to calculate fitted adjusted reading speeds for each available print size.

A logistic model equation is used to create the reading speed function ƒ(xⱼ), formulated as

$$\frac{ϕ}{1 + exp [ - a ( {x}_{j} - b ) ]} .$$

yⱼ can be set as the adjusted reading speed at the corresponding print size, xⱼ. yⱼ substantially characterizes the reading curve which the reading speed model according to the present invention intends to produce.

As already explained, yⱼ can be assumed to have a normal distribution according to the following exemplary expression:

$${y}_{j} ∼ N ( \frac{ϕ}{1 + exp [ - a ( {x}_{j} - b ) ]} , σ )$$

wherein the mean is defined by the above logistic model equation.

Parameter values of ϕ, a, and b of the logistic model equation that maximize the log posterior distribution are then used as updated estimates for ϕ, a, and b. In general terms, the optimization of the log posterior distribution has been described in the disclosure of the invention.

Stopping criteria for the iterations of adaptively fitting the provided reading speed model to received observation data are based on cutoff values for the posterior standard deviation of the maximum reading speed, or MRS, and for the critical print size, or CPS.

The posterior standard deviation of these metrics are calculated based on a number of samples from the approximate normal posterior distribution. The Hessian matrix of the log-posterior distribution is numerically estimated, and the samples are drawn from a multivariate normal distribution with the mean being the parameter estimates, and the covariance matrix being the inverse of the Hessian matrix. For each of the draws of the parameters, MRS and CPS are calculated. The posterior standard deviation of these metrics would then be the standard deviation of all MRS and CPS values. The stopping criteria can depend on some combination of the standard errors for MRS and CPS, or on just one of the standard errors.

If the stopping criteria are not met, subsequent administrations of reading runs of the reading test to the individual are envisaged, wherein the print size of the next/subsequently administered printed stimulus is controlled, so as to achieve that a corresponding point is obtained on the reading curve in a targeted position.

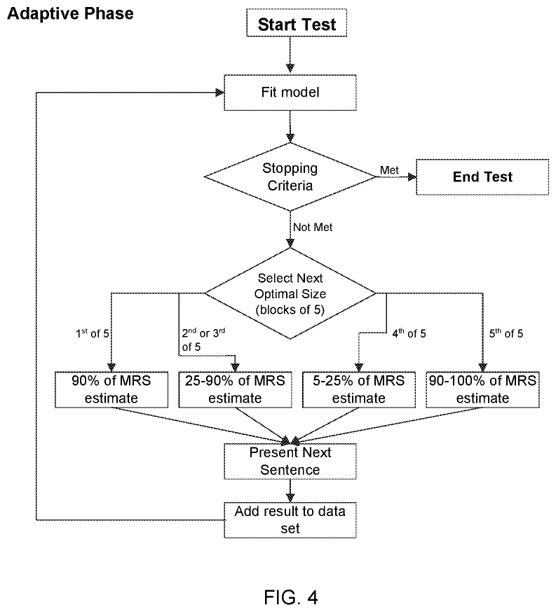

As evident from FIG. 4, the print size of the next/subsequently administered printed stimulus is adaptively selected in blocks of five distinct print size values, so that corresponding, distinct points are obtained in respective targeted positions of the reading curve, such as on an elbow, on a slope, and/or on a plateau of the reading curve. Alternatively, blocks of three distinct print sizes have been further tested.

These targeted positions can correspond to respective percentages of the maximum reading speed, or MRS. By way of example, a first print size can be selected corresponding to 90% of the MRS estimate, in order to obtain a point on the elbow of the reading curve. A second and/or third print size can be selected corresponding to a range of 25%-90% of the MRS estimate, to obtain respective points on an upper portion of the slope of the reading curve. A fourth print size can be chosen corresponding to a range of 5%-25% of the MRS estimate, so as obtain a point on a lower portion of the slope of the reading curve. Finally, a fifth print size can be selected corresponding to a range of 90%-100% of the MRS estimate to produce a point on a plateau of the reading curve.

Other schemes can be considered for adaptively locating the next optimal point. For instance, an adaptive scheme can allocate the next optimal point at the current estimate of some print size corresponding to simply one set percentage of the maximum reading speed, or MRS. For example, the print size corresponding to 80% of the MRS could be the next optimal print size for each iteration.

A different adaptive scheme can alternate between different percentages of MRS, based on what iteration number of the adaptive scheme has been reached. An example of this approach would let the next optimal print size vary between the print sizes corresponding to 99% of the MRS, 75% of the MRS, and 15% of the MRS. When the number of iterations is divided by four, the remainder determines what the next optimal print size is. If the remainder is zero, the next optimal print size is the print size corresponding to 99% of the MRS. If the remainder is one or two, the next optimal print size is that print size corresponding to 75% of the MRS, and if the remainder is three, the next optimal print size is the print size corresponding to 15% of the MRS. The aforementioned three percentages can be fixed or can be made to vary.

Also, a print size can be added corresponding to a point on the plateau of the reading curve, by choosing a print size that falls between the current estimate of CPS and the maximum print size. This can be helpful to obtain a more accurate estimate of MRS.

The last step in the adaptive cycle generates an adjusted reading speed at the optimal point and adds it to the final data set for the adaptive phase.

Once the function has cycled through the iterations of the adaptive phase until some stop criteria is met, it adds the observation to the final data set. The adaptive phase function returns the fits from each iteration of the adaptive phase, and the final data set from the adaptive phase.

Other aspects of the disclosed embodiments will be apparent to those skilled in the art from consideration of the specification and practice of the disclosed embodiments. It is intended that the specification and examples be considered as exemplary only, with exemplary scopes of the disclosed embodiments being indicated by the following exemplary claims comprising examples and embodiments of the invention.

1. A computer implemented method for estimating a reading speed curve of an individual, comprising the steps of:
at a computing device including one or more processors and one or more input and/or output elements,
providing a reading speed model, based on a reading speed function comprising a set of parameters and a set of metrics;
determining a printed stimulus to be presented to the individual by way of a run of a reading test and determining one or more stimulus features associated therewith;
controlling an administration of the run of the reading test to the individual based on the one or more determined stimulus features;
wherein the reading speed function is configured to characterize the reading speed of the individual over a range of the one or more stimulus features of the presented printed stimulus, thus representing a reading curve for the individual, and to mimic at least part of a run of the administered reading speed test, and
receiving reading speed observation data based on the presented printed stimulus and corresponding to one or more responses made by the individual in the reading test;
wherein the method comprises an adaptive phase of:
fitting the reading speed model to the received observation data such that the set of parameters and/or of metrics of the reading speed model and/or stimulus features is updated based on the reading speed observation data, to update the estimated reading speed of the individual;
iterating the providing, the determining, the controlling, the receiving and the fitting according to stopping criteria to adaptively refine the estimated reading speed for the individual for a plurality of subsequent administrations of reading runs of the reading test;
characterized in that
before the adaptive phase, providing the reading speed model comprises a step of mapping out the reading speed function for the currently tested individual, by executing a preliminary administration of reading runs of the reading test to the individual based on presentation of printed stimuli having predetermined values of the one or more features; and by consequently receiving reading speed data to obtain a corresponding number of points on the reading curve.

2. The computer implemented method of claim 1, wherein the presented printed stimulus is a sentence.

3. The computer implemented method of claim 1, wherein providing the reading speed model comprises a step of defining the range of the one or more stimulus features and identifying a pre-defined number of predetermined values within the range of the one or more stimulus features to divide the range into corresponding portions.

4. The computer implemented method of claim 3, wherein the portions of predetermined values are substantially equal.

5. The computer implemented method of claim 1, wherein one of the features of the presented printed stimulus is print size.

6. The computer implemented method of claim 5, wherein providing the reading speed model comprises a step of calculating, by the reading speed function, adjusted reading speeds for each of the identified predetermined values of print size; and of counting the number of errors which the individual makes when reading at each of the identified predetermined values of print size.

7. The computer implemented method of claim 6, wherein the reading speed function corresponds to a logistic reading speed model, according to the following distribution
$$f ( {x}_{j} ) = \frac{ϕ}{1 + exp [ - a ( {x}_{j} - b ) ]}$$
wherein:
ϕ is a metric corresponding to the maximum reading speed on the reading curve for the individual (that is, the individual's reading speed when reading is not limited by print size);
a is the rate of change from 0 to ϕ on the reading curve; and
b is the inflection point of the reading curve.

8. The computer implemented method of claim 7, wherein providing the reading speed model comprises a step of testing the individual by presenting printed stimuli starting at the largest predetermined value of print size of the range and a step of receiving reading speed observation data to obtain a first observed value of the initial reading speed, thus obtaining a point on the reading curve corresponding to such largest predetermined value print size of the range.

9. The computer implemented method of claim 8, wherein providing the reading speed model comprises a step of providing check criteria based respectively on a cutoff error value for the number of errors which the individual makes at a given print size value; and on a cutoff speed value for the adjusted reading speed at a given print size value.

10. The computer implemented method of claim 9, wherein the cutoff speed value for the adjusted reading speed at a given print size value is a percentage of the first observed value of the initial reading speed.

11. The computer implemented method of claim 10, wherein providing the reading speed model comprises a step of comparing respectively the number of errors which the individual makes at a given print size value to the cutoff error value, wherein a first check criterion is met if the number of errors is less than or equal to the cutoff error value; and of comparing the adjusted reading speed at a given print size value to the cutoff speed value, wherein a second check criterion is met if the adjusted reading speed is equal or above the cutoff speed value.

12. The computer implemented method of claim 11, wherein providing the reading speed model comprises, if both check criteria are met, a step of reducing the print size value to the next smaller predetermined value; a step of testing the individual by presenting printed stimuli at the next smaller predetermined value of print size; and a step of comparing respectively the number of errors which the individual makes at the next smaller predetermined value of print size to the cutoff error value; and of comparing the adjusted reading speed at the next smaller predetermined value of print size to the cutoff speed value.

13. The computer implemented method of claim 12, wherein, if no further smaller predetermined value of print size exists, the reading speed observation data and the errors made by the individual when reading at each of the identified predetermined values of print size are stored for completing the provision of the reading speed model.

14. The computer implemented method of claim 11, wherein providing the reading speed model comprises, if either of the check criteria is not met, the step of increasing the print size value by a predefined amount with respect to the current print size value, if possible within the range of predetermined values of print sizes; the step of testing the individual by presenting printed stimuli at the corresponding increased print size value; and of comparing respectively the number of errors which the individual makes at the corresponding increased print size value to the cutoff error value, and of comparing the adjusted reading speed at corresponding increased print size value to the cutoff speed value.

15. The computer implemented method of claim 14, comprising the step of, if both check criteria are met, storing the reading speed observation data and the errors made by the individual when reading at each of the values of print size, for completing the provision of the reading speed model; otherwise, if either of the check criteria is not met, comprising the step of further increasing the print size value by a predefined amount with respect to the current print size value, up to the last print size value wherein both check criteria were met.

16. The computer implemented method of claim 14, comprising the step of repeating the testing of the individual at the largest predetermined value of print size of the range, if either of the check criteria is not met already when testing the individual by presenting printed stimuli starting at the largest predetermined value of print size of the range; and, if either of the check criteria is again not met, comprising the step of not proceeding to the adaptive phase.

17. The computer implemented method of claim 16, comprising the step of, irrespective of observation scenario/outcome whether the check criteria were met or whether the check criteria were not met, storing the reading speed observation data and the errors made by the individual when reading at each of the values of tested print sizes in a final data set, in order to complete the provision of the reading speed model.

18. The computer implemented method of claim 17, wherein the first iteration step of adaptively fitting the provided reading speed model to received observation data is based on fitting the provided reading speed model to the observation data.

19. The computer implemented method of claim 18, wherein adaptively fitting the provided reading speed model to received observation data corresponding to subsequent administrations of reading runs of the reading test to the individual comprises the step of defining a fitting function, comprising the reading speed function, and the step of optimizing the log posterior for the defined fitting function, to adaptively estimate the set of parameters and the set of metrics of the reading speed model.

20. The computer implemented method of claim 19, wherein the set of parameters and the set of metrics of the reading speed model thus estimated are used to calculate fitted adjusted reading speeds for each available print size.

21. The computer implemented method of claim 1, wherein the stopping criteria for the iterations of adaptively fitting the provided reading speed model to received observation data are based on cutoff values for estimated metrics, preferably on the posterior standard deviation, or standard errors, or the respective estimates thereof or on a combination of the standard errors.

22. The computer implemented method of claim 21, wherein the stopping criteria are based on cutoff values for the maximum reading speed, or MRS, and for the critical print size, or CPS.

23. The computer implemented method of claim 1, wherein adaptively fitting the provided reading speed model to received observation data corresponding to subsequent administrations of reading runs of the reading test to the individual comprises the step of controlling the print size of the next/subsequently administered printed stimulus, so that a corresponding point is obtained on the reading curve in a targeted position.

24. The computer implemented method of claim 23, wherein choosing the corresponding point on the reading curve lies within a subset of a parameter or metric space for one of the parameters or metrics of the reading speed model, such as for the maximum reading speed, or MRS.

25. The computer implemented method of claim 24, wherein the print size of the next/subsequently administered printed stimulus is selected in blocks of five distinct print size values, so that corresponding, distinct points are obtained in respective targeted positions of the reading curve, such as on an elbow, on a slope, and/or on a plateau of the reading curve, preferably corresponding to respective percentages (e.g. 90%; 25%-90%; 5%-25%, 90%-100%) of the maximum reading speed, or MRS.

26. The computer implemented method of claim 25, wherein the one or more responses made by the individual in the reading test are assessed by voice recognition software, both for reporting reading time and for counting errors.

27. A system comprising:
a computing device including:
one or more processors;
one or more input and/or output elements;
memory; and
one or more programs stored in the memory, the one or more programs including instructions for:
providing a reading speed model, based on a reading speed function comprising a set of parameters and a set of metrics,
determining a printed stimulus to be presented to the individual by way of a reading run of a reading test and determining one or more stimulus features associated therewith;
controlling an administration of the run of the reading test to the individual based on the one or more determined stimulus features;
wherein the reading speed function is configured to characterize the reading speed of the individual over a range of the one or more stimulus features of the presented printed stimulus, thus representing a reading curve for the individual, and to mimic at least part of a run of the administered reading speed test, and
receiving reading speed observation data based on the presented printed stimulus and corresponding to one or more responses made by the individual in the reading test;
the one or more programs comprising an adaptive phase including instructions for:
adaptively fitting the reading speed model to the received observation data such that the set of parameters and/or of metrics of the reading speed model and/or stimulus features is updated based on the reading speed observation data, to update the estimated reading speed of the individual;
iterating the providing, the determining, the controlling, the receiving and the fitting according to stopping criteria to adaptively refine the estimated reading speed for the individual for a plurality of subsequent administrations of reading runs of the reading test;
the one or more programs comprising instructions, to be executed before the adaptive phase, for mapping out the reading speed function for the currently tested individual in order to provide the reading speed model, by executing a preliminary administration of reading runs of the reading test to the individual based on presentation of printed stimuli having predetermined values of the one or more features; and by consequently receiving reading speed data to obtain a corresponding number of points on the reading curve.

28. A non-transitory computer-readable storage medium storing one or more programs configured to be executed by one or more processors of an electronic device with one or more input and/or output elements, the one or more programs including instructions for:
providing a reading speed model, based on a reading speed function comprising a set of parameters and a set of metrics;
determining a printed stimulus to be presented to the individual by way of a reading run of a reading test and determining one or more stimulus features associated therewith;
controlling an administration of the run of the reading test to the individual based on the one or more determined stimulus features;
wherein the reading speed function is configured to characterize the reading speed of the individual over a range of the one or more stimulus features of the presented printed stimulus, thus representing a reading curve for the individual, and to mimic at least part of a run of the administered reading speed test, and
receiving reading speed observation data based on the presented printed stimulus and corresponding to one or more responses made by the individual in the reading test;
the one or more programs comprising an adaptive phase including instructions for:
adaptively fitting the reading speed model to the received observation data such that the set of parameters and/or of metrics of the reading speed model and/or stimulus features is updated based on the reading speed observation data, to update the estimated reading speed of the individual;
iterating the providing, the determining, the controlling, the receiving and the fitting according to stopping criteria to adaptively refine the estimated reading speed for the individual for a plurality of subsequent administrations of reading runs of the reading test;
the one or more programs comprising instructions, to be executed before the adaptive phase, for mapping out the reading speed function for the currently tested individual in order to provide the reading speed model, by executing a preliminary administration of reading runs of the reading test to the individual based on presentation of printed stimuli having predetermined values of the one or more features; and by consequently generating and receiving reading speed data to obtain a corresponding number of points on the reading curve.
